(19) United States
(12) Patent Application Publication
(10) Pub. No.: US 2008/0167687 A1
(43) Pub. Date: Jul. 10, 2008

(54) DYNAMIC LINKING MEMBER FOR SPINE STABILIZATION SYSTEM

(76) Inventors: Dennis Colleran, North Attleboro, MA (US); Michael Perriello, Hopedale, MA (US); Arnold Oyola, Northborough, MA (US)

Correspondence Address: CARR LLP (IST), 670 FOUNDERS SQUARE, 900 JACKSON STREET, DALLAS, TX, 75202, US

(21) Appl. No.: 11/969,165

(22) Filed: Jan. 3, 2008

Related U.S. Application Data
(60) Provisional application No. 60/883,314, filed on Jan. 3, 2007.

(51) Int. Cl. A61B 17/58

(52) U.S. Cl. 606/257; 606/246

(57) ABSTRACT

An apparatus for stabilizing a spine is disclosed which includes a first link member pivotably coupled to a second link member. The first or second link members may have one or more stops to limit the motion of the implant. The first and second link members may include a first and second respective height adjustment mechanisms. A force control mechanism may also be provided which is coupled to the implant and includes a main body coupled to an extension control member and a flexion control member. The extension control member may extend from the main body towards the first stop and the flexion control member extends from the main body towards the second stop.

### CROSS REFERENCE TO RELATED APPLICATIONS

This application relates to, and claims the benefit of the filing date of: co-pending U.S. provisional patent application Ser. No. 60/883,314 entitled “Dynamic Linking Member for Spine Stabilization System” filed Jan. 3, 2007 the entire contents of which are incorporated herein by reference for all purposes. This application is also commonly owned with U.S. application Ser. No. 11/467,798, filed on Aug. 28, 2006, entitled “Alignment Instrument for Dynamic Spinal Stabilization Systems; Ser. No. 11/443,236, filed on May 30, 2006, entitled “System and Method for Dynamic Skeletal Stabilization”; Ser. No. 11/303,138, filed on Dec. 16, 2005, entitled “Three Column Support Dynamic Stabilization System and Method; Ser. No. 60/825,078, filed on Sep. 8, 2006, entitled “Offset Adjustable Dynamic Stabilization System”; Ser. No. 60/826,807, filed on Sep. 25, 2006, entitled “Offset Adjustable Dynamic Stabilization System”; Ser. No. 60/826,817, filed on Sep. 25, 2006, entitled “Offset Adjustable Dynamic Stabilization System”; Ser. No. 60/863,284, filed on Oct. 27, 2006, entitled “Alignment Instrument for Dynamic Spinal Stabilization Systems”; Ser. No. 60/826,763, filed on Sep. 25, 2006, entitled “Alignment Instrument for Dynamic Spinal Stabilization Systems”; Ser. No. 60/786,898, filed on Mar. 29, 2006, entitled “Full Motion Spherical Linkage Implant System”; Ser. No. 60/831,879, filed on Jul. 19, 2006, entitled “Locking Assembly” Ser. No. 60/793,829, filed on Apr. 21, 2006, entitled “Micro Motion Spherical Linkage Implant System”; Ser. No. 60/814,753, filed on Jun. 19, 2006, entitled “Multi-Level Spherical Linkage Implant System”; Ser. No. 10/914,751, filed on Aug. 9, 2004, entitled “System and Method for Dynamic Skeletal Stabilization”, the disclosures of which are incorporated herein by reference.

### TECHNICAL FIELD

The invention relates in general to spine stabilization, and in particular to dynamic spine stabilization systems.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis, into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered around an elliptical center of rotation. During such rotation, the anterior (front) edges of neighboring vertebrae move closer together, while the posterior edges move farther apart, compressing the anterior of the spine Similarly, during extension, the posterior edges of neighboring vertebrae move closer together, while the anterior edges move farther apart, compressing the posterior of the spine. Also during flexion and extension, the vertebrae move in horizontal relationship to each other, providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through roughly an arc of 10° relative to its neighbor vertebrae, throughout right and left lateral bending.

Rotational movement about a vertical axis relative to the portion of the spine moving is also desirable. For example, rotational movement can be described as the clockwise or counter-clockwise twisting rotation of the vertebrae, such as during a golf swing.

The inter-vertebral spacing (between neighboring vertebrae) in, a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress or become degenerated. The compressed or degenerated disc may cause pressure to be exerted on nerves extending from the spinal cord by this reduced inter-vertebral spacing. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other, thereby maintaining space for the nerves to exit without being impinged upon by movements of the spine.

In one such procedure, screws are embedded in adjacent vertebrae pedicles and rigid rods or plates are then secured between the screws. In such a situation, the pedicle screws (which are in effect extensions of the vertebrae) then press against the rigid spacer which serves to distract the degenerated disc space, maintaining adequate separation between the neighboring vertebrae so as to prevent the vertebrae from compressing the nerves. This prevents nerve pressure due to extension of the spine; however, when the patient then tries to bend forward (putting the spine in flexion), the posterior portions of at least two vertebrae are effectively held together. Furthermore, the lateral bending or rotational movement between the affected vertebrae is significantly reduced due to the rigid connection of the spacers. Overall movement of the spine is reduced as more vertebrae are distracted by such rigid spacers. This type of spacer not only limits the patient's movements, but also places additional stress on other portions of the spine (typically, the stress placed on adjacent vertebrae without spacers being the worse), often leading to further complications at a later date.

Current dynamic spinal implant systems do not control vertebral movement about all three axis to emulate a healthy spine. Current systems also do not offer a force control mechanism that works in conjunction with a spinal implant system that controls movement about all three axis to emulate a healthy spine. For a dynamic spinal implant system to be oriented properly the height of the implant, or the distance from an area between the spinal disc to the spinal implant may need to be adjusted. Current systems do not allow for this height adjustment of the spinal implant in-between two pedicle screws.

These and other features, and advantages, will be more clearly understood from the following detailed description taken in conjunction with the accompanying drawings. It is important to note the drawings are not intended to represent the only aspect of the invention. Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a top view of one possible embodiment of a dynamic linking implant that may be incorporated in a dynamic stabilization system;

FIG. 2 is an oblique view of one possible embodiment of a linking member of that may be incorporated in the dynamic linking implant shown in FIG. 1;

FIG. 3 is an oblique view of one possible embodiment of a second linking member that may be incorporated in the dynamic linking implant shown in FIG. 1;

FIG. 4 is an enlarged cross sectional side view of the linking members shown in FIG. 1;

FIG. 5 is an oblique view of one possible embodiment of a force control mechanism that may be incorporated in linking system shown in FIG. 1;

FIG. 6 is an detailed top view of dynamic linking implant shown in FIG. 1;

FIG. 7 is an oblique view of one possible embodiment of a height adjustment bracket that may be incorporated into the dynamic linking implant shown in FIG. 1;

FIG. 8 is an enlarged oblique view of the height adjustment bracket of FIG. 7 mated to one of the linking members of the dynamic linking implant shown in FIG. 1;

FIG. 9 is a cross sectional view of one possible embodiment of a height adjustment mechanism that may be incorporated into the dynamic linking implant shown in FIG. 1; and

FIG. 10 is an oblique view of another possible embodiment of a dynamic stabilization system.

### DETAILED DESCRIPTION

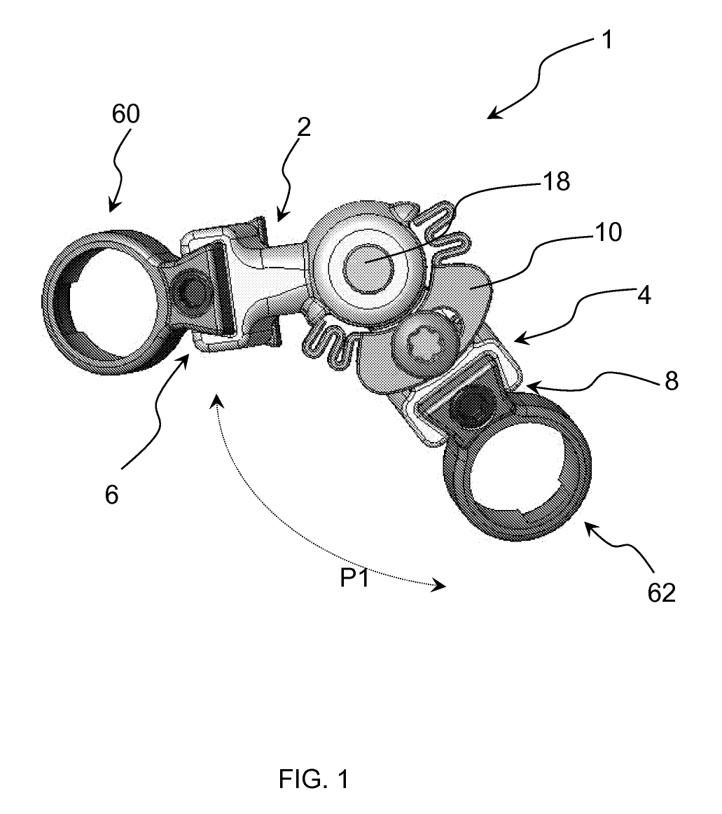

Referring now to FIG. 1, a top view of one possible embodiment of a dynamic linking implant 1 is illustrated, which may be incorporated into a dynamic stabilization system (not shown). The dynamic linking implant 1 may incorporate a first linking member 2, a second linking member 4, a force control mechanism 10 and one or more height adjustment mechanisms 6 and 8. The first linking member 2 may be pivotably coupled to the second linking member 4 with a pin 18 which may control movement of the dynamic linking implant 1 along a curved path P1. The dynamic linking implant 1 may be coupled to one or more bone anchors (not shown) which may then couple to a portion of a spine, such as a vertebra. The height adjustment mechanisms 6 and 8 may include a bracket which may couple the dynamic linking implant 1 to a pedicle screw (not shown).

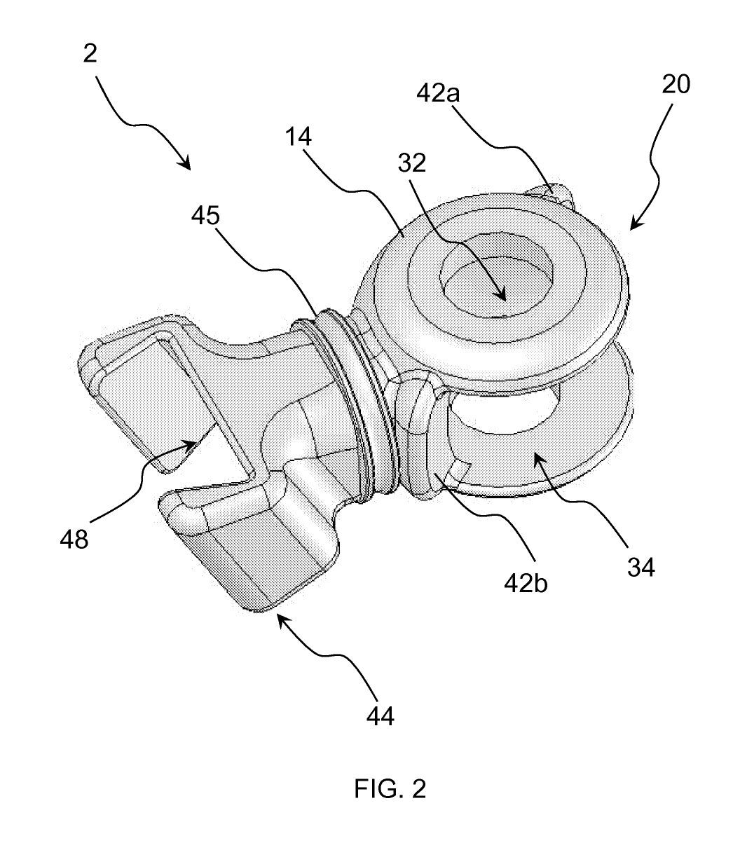

Referring to FIG. 2 the first linking member 2 may extend along a curved longitudinal axis and may have a first shaped end 20. The first shaped end 20 may have a generally cylindrical shape with a top and bottom surface and a spherical outer side surface 14. A bore 32 may extend through the top and bottom surfaces of first linking member 2. A slot 34 may extend into the side surface of the first linking member 2. The slot 34 may be defined by a spherical inner wall. One or more projections 42a and 42b may extend radially outward from the spherical outer side surface of first shaped end 20 and may be circumferentially spaced apart from each other. As will be described in greater detail below, projections 42a and 42b may act as a rotational stop to limit movement of the dynamic linking implant 1.

The first linking member 2 may have a second shaped end 44 connected to first shaped end 20. A groove or attachment feature 45 may be positioned between the first shaped end 20 and the second shaped end 44. The feature 45 may aid in attachment of a cover (not shown). The second shaped end 44 may have a generally rectangular shape having a top surface, a bottom surface. A passage 48 may extend through the top and bottom surfaces of the second shaped end 44. The passage 48 may have a dovetail shape with an open front section and two non parallel side walls. As will be described in greater detail below the passage 48 of the second shaped end 44 may be dimensioned to mate with the height adjustment mechanism 6.

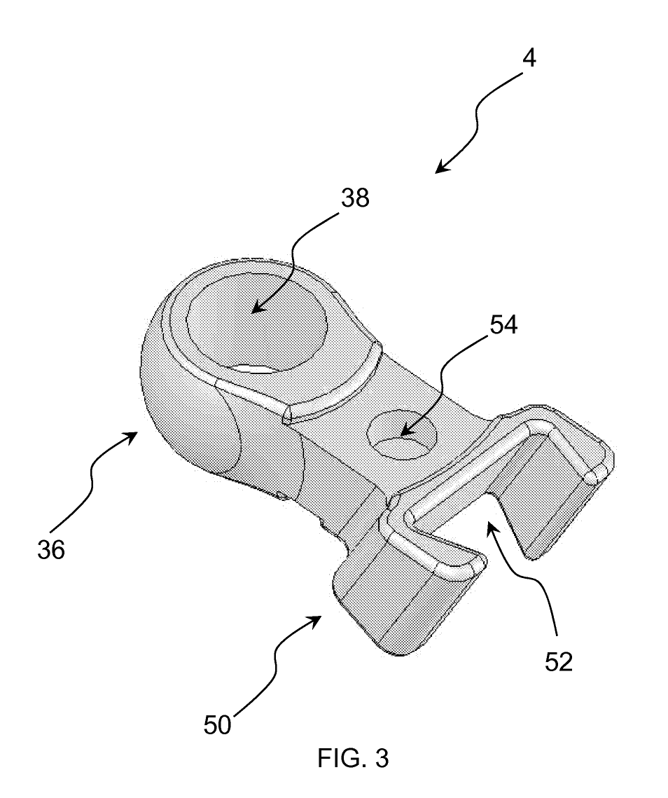

Referring to FIG. 3, one embodiment of a second linking member 4 is shown. The second linking member 4 may extend along a curved longitudinal axis and may have a first shaped end 36. In certain embodiments, the first shaped end 36 may have a generally cylindrical shape with a top and bottom surface and a spherical outer side surface. The second linking member 4 may have a groove or attachment features (not shown) similar to the attachment feature 45 shown in FIG. 2, for attaching a cover (not shown). A bore 38 may extend through the top and bottom surfaces of second linking member 4.

In certain embodiments, the second linking member 4 may have a second shaped end 50. The second shaped end 50 may be connected to the first shaped end 36. The second shaped end 50 may have a generally rectangular shape with a top surface and a bottom surface. A passage 52 may extend through the top and bottom surfaces of the second shaped end 44. The passage 52 may have a dovetail shape with an open front section and two non parallel side walls. As will be described in greater detail below the passage 52 of the second shaped end 50 may be dimensioned to mate with the height adjustment mechanism 8. In the present example, the top surface of second linking member 4 may have a hole 54 that is located between the first shaped end 36 and second shaped end 50. In certain embodiments the hole 54 may have a threaded internal surface which may couple to an adjustment member (not shown) of the a force control mechanism of FIG. 1.

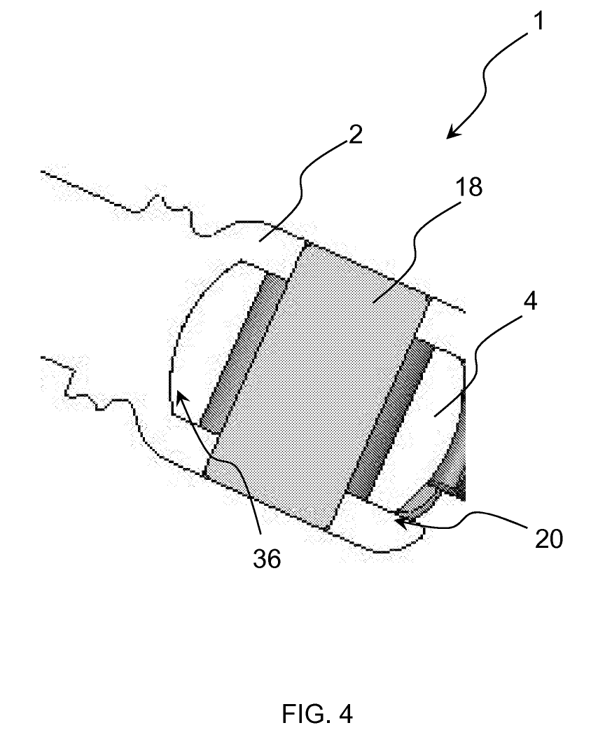

Referring now to FIG. 4, a detailed cross sectional view is shown of the dynamic linking implant 1 illustrating the first linking member 2 coupled to the second linking member 4. The first shaped end 36 (see also FIG. 3) of the second linking member 4 may fit within the slot of 34 (see FIG. 2) of the first shaped end 20 of the first linking member 2. The spherical outer side surface of the first shaped end 36 (see FIG. 3) may be dimensioned to rotate within the slot 34 of the first linking member 2. The first shaped end 20 of the first linking member 2 and the first shaped end 36 of the second linking member 4 may be aligned such that the central axis of bore 32 of first linking member is aligned with bore 38 of second linking member 4. After the first shaped end 20 of the first linking member 2 and the first shaped end 36 of the second linking member 4 are properly aligned, the pin 18 may be inserted through bores 32 and 34 to secure the first linking member 2 to second linking member 4. Once the first linking member 2 is secured to the second linking member 4, both linking members may be able to rotate about pin 18 as shown by path P1 in FIG. 1.

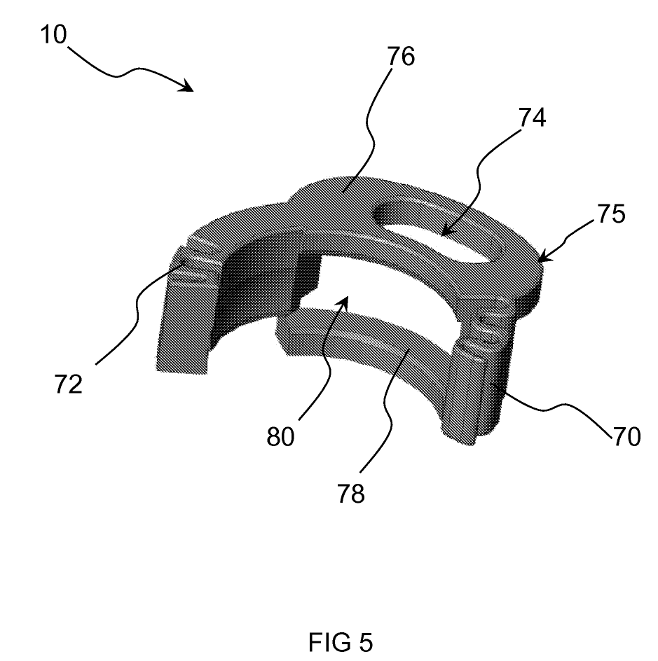

FIG. 5 illustrates one possible embodiment of the force control mechanism 10 that may be incorporated to control or limit the force required for the first linking member 2 and the second linking member 4 to rotate relative to each other. The force control mechanism 10 may include a main body 75 with a top wall 76, a bottom wall 78 and an open space 80 between the top and bottom walls 76 and 78. In certain embodiments, the force control mechanism 10 may have a slot 74 that extends through its top wall 76. The top and bottom walls 76 and 78 may be connected by two side walls which may have one or more dampening members 70 and 72. The dampening members 70 and 72 may act as flexion and extension control members to control or limit the force of the dynamic linking implant during flexion or extension of a spine. The dampening members 70 and 72 may include a plurality of successive waves in which the waves include alternating curved crest and curved trough portions. The dampening members 70 and 72 may achieve their dampening characteristics through the wave-like design. In the present example dampening members 70 and 72 may extend along a curved or arcuate longitudinal axis, but may also extend in a linear fashion. The space 80 in-between the top 76 and bottom 78 walls of the force control mechanism 10 may be dimensioned to receive the first shaped end 36 of second linking member 4, as shown in FIG. 1.

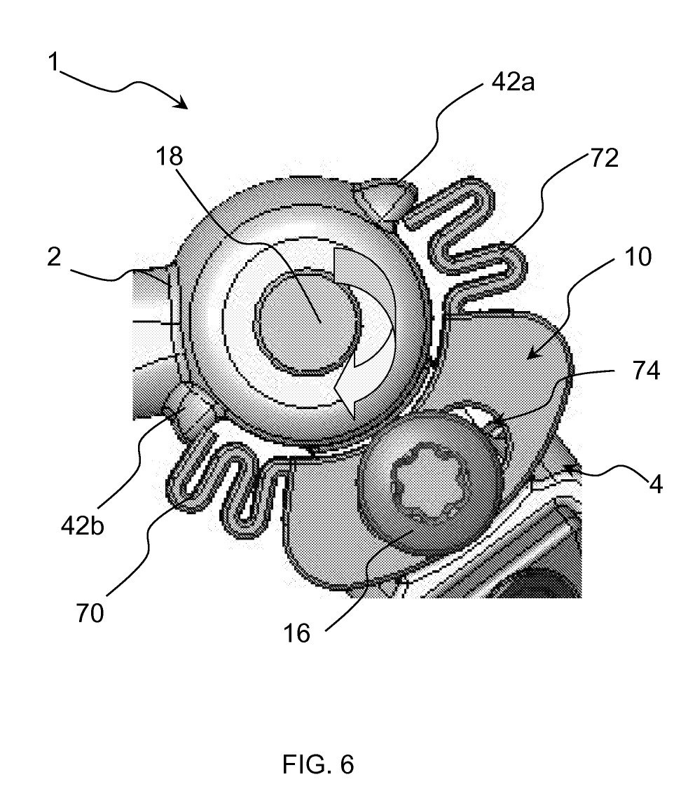

FIG. 6 shows an enlarged top view of the dynamic the dynamic linking implant 1 illustrating the force control mechanism 10 assembled to the first and second linking members 2 and 4. The slot 74 of the force control mechanism 10 may align with the hole 54 (see FIG. 3) of the second linking member 4. An extension or flexion force of the force control mechanism 10 may be adjusted by adjusting the position of the slot 74 relative to the hole 54. An adjustment member 16 may be positioned within the slot 74 and the hole 54 to secure the position of the force control mechanism. The dampening member 72 may extend from the main body 75 towards the first protrusions 42a and the dampening member 70 may extend from the main body towards the second protrusion 42b. A distal end portion of dampening members 72 and 70 may contact protrusions 42a and 42b (respectively) of the first linking member 2. The protrusions 42a and 42b may act as stops or limits to prevent flexion or extension of the spine by limiting the movement of the dynamic linking implant 1.

The dampening members 72 and 70 may exert a force against protrusions 42a and 42b, respectively as a spine moves in flexion or extension. As the first and second linking members 2 and 4 move in a first direction (as shown by large arrow in FIG. 6), one dampening member 70 may compress against protrusion 42b, while the other dampening member 72 may relax or extend, to a neutral position as shown in FIG. 6. The dampening member 72 may compress and exert a force against protrusion 42a, if the first and second linking members 2 and 4 are moved in the opposite direction. The amount of force exerted on protrusions 42a and 42b by dampening members 72 and 70 (respectively) may be adjusted by adjusting the position of slot 74 relative to member 16. For example, if the adjustment member 16 is positioned further away from one end of slot 74, as shown in FIG. 6 then the dampening member 70 may be positioned closer to the protrusion 42a and thus may compress more (and member 70 may be compressed less) than if member 16 was positioned in the middle (or at the other end) of slot 74. In certain embodiments the force control mechanism 10 may be a unitary component or an assembly that is machined from a metallic material such as nitinol, stainless steel or titanium. Alternatively, the force control mechanism 10 may be molded or machined from an elastomeric or polymeric material.

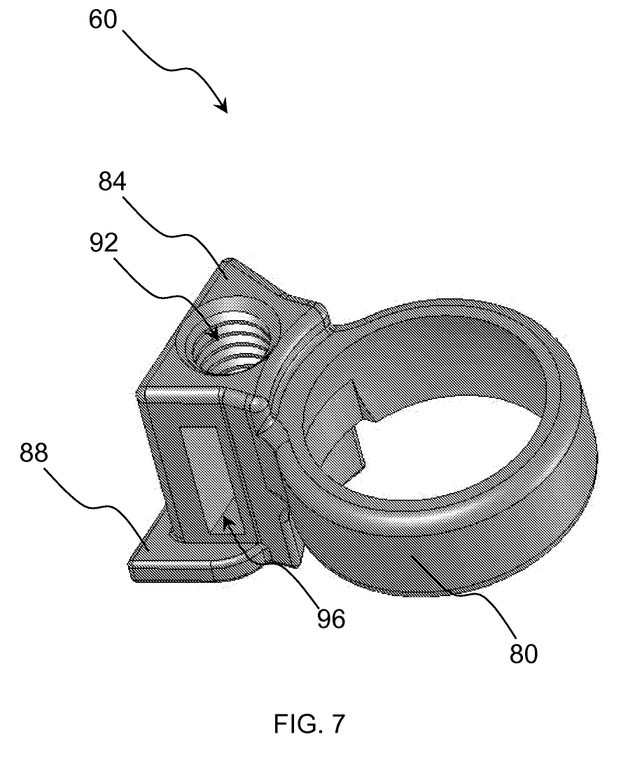

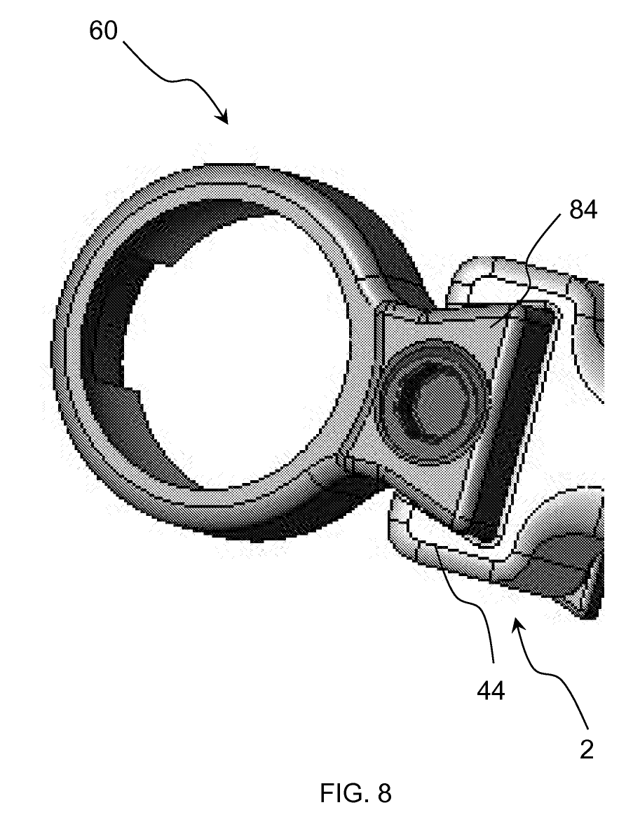

In certain embodiments, the height adjustment mechanism 6 and 8 may include the brackets 60 and 62 which may incorporate various features to adjust and/or secure brackets 60 and 62 to linking members 2 and 4. The brackets 60 and 62 may be identical in structure and function, thus only the bracket 60 will be described in detail. Referring to FIGS. 7 and 8, one embodiment of the height adjustment bracket 60 is shown. The height adjustment bracket 60 may be incorporated into one or more height adjustment mechanisms as shown in FIG. 1. The adjustment brackets 60 may have a ring shaped first end 80 that is generally cylindrically shaped with an aperture extending through its center axis. The ring shaped end 80 may allow for the dynamic linking implant 1 to be connected to a vertebrae (or other bone) through various bone anchoring means, such as a pedicle screw (not shown). The adjustment bracket 60 may have a second shaped end 84 that has a dovetail geometry which may correspond to the geometry of passage 48 (see FIG. 2) of the first linking member 2. The second shaped end 84 may be couple to a plate member 88. The second shaped ends 44 of the first linking member 2 may slide over the second shaped ends 84 of adjustment brackets 60 as shown in FIG. 8. The plate 88 may prevent the first linking member 2 from sliding off bracket 60. The brackets 60 may have a hole 92 that extends into the top surfaces of second shaped end portions 84. A distal end section of hole 92 may be in communication with a side slot 96 which may extend into a side wall of second shaped end portion 92.

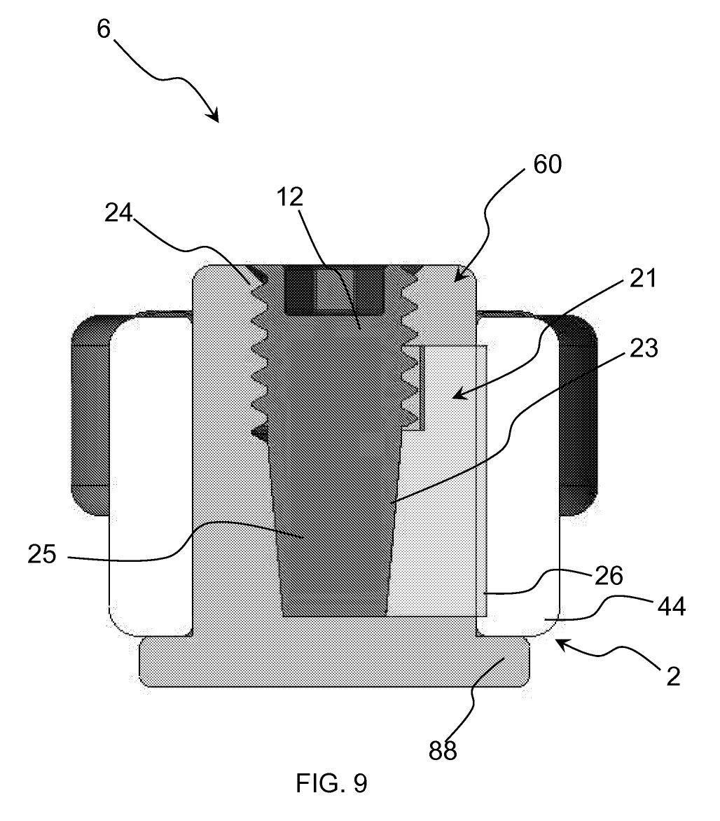

Referring to FIG. 9, a cross sectional side view of one embodiment of the height adjustment mechanism 6 is shown. The height adjustment components for height adjustment mechanism 6 may be identical for height adjustment mechanism 8 and thus will not be repeated. A wedge member 21 may be placed within the respective side slot 96 as shown in FIG. 8. The wedge member 21 may have a first tapered side wall 23 which faces the hole 92 (see also FIG. 7). The hole 92 may have an upper threaded section 24 that mates with a locking member 12. A distal end portion of the hole 92 may have tapered wall(s) which may correspond to a tapered distal end section 25 of the locking member 12. As the locking member 12 is inserted into hole 92 the tapered section 25 may contact the tapered side wall 23 of the wedge member 21. As locking member 12 is inserted further into the hole 92, the wedge member 21 may be forced in an outward direction so that wedge member 21 contacts and exerts a force against an inner side wall 26 of the second end portion 44 of the first linking member 2. The wedge member 21 may secure the first linking member 2 to the bracket 60. The bottom surface of the second shaped end portion 48 may contact the plate of the bracket 60. Alternatively, a gap may be located between the plate 88 and the bottom surface of second shaped end portion 44, depending on the desired final position of the dynamic linking implant. The second shaped end portion 48 may be raised or lowered in relation to the bracket 60 until the wedge member 21 is locked into place by the locking member 12.

As previously described above, the position or height of the brackets 60 and 62 may be adjusted relative to the linking members 2 and 4. The height adjustment mechanism 6 and 8 may allow the dynamic linking implant 1 to be adjusted independently of a bone anchor, such as a pedicle screw, to which the dynamic implant 1 is coupled to. There may be several drawbacks to a surgeon adjusting the height of an implant by changing the depth of a pedicle screw. First, the pedicle screw may loosen from the bone if the screw is not inserted to a certain depth and second if the pedicle screw is inserted to deep into the pedicle the screw may exit the pedicle and impinge or damage neighboring anatomy.

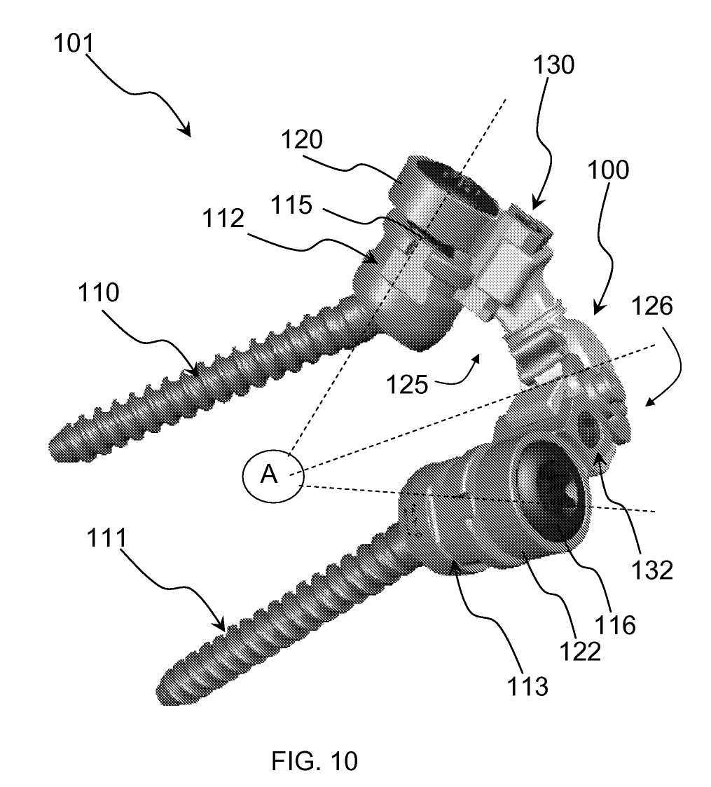

Turning to FIG. 10, an alternative embodiment of a dynamic linking implant 100 is shown as part of a dynamic stabilization system 101. The dynamic linking implant 100 may be similar in structure and function as the dynamic linking implant 1 described above. The dynamic stabilization system 101 may include the dynamic linking implant 100 coupled to a pair of bone anchors bone anchors, such as pedicle screws 110 and 111. The pedicle screws 110 and 111 may each have a polyaxial head 112 and 113 which may aid in coupling the dynamic linking implant 100 to the pedicle screws 110 and 111. The pair of pedicle screws 110 and 111 may be inserted into a pair of adjacent vertebrae (not shown). The dynamic stabilization implant 100 may then be coupled to the respective polyaxial heads 112 and 113. In certain embodiments the polyaxial heads 112 and 113 may have a post 115 and 116 which may receive a portion of the dynamic stabilization implant 100, such as the bracket 120 and 122. In other embodiments the polyaxial heads 112 and 113 may have a slot to receive a rod portion of the dynamic stabilization implant (not shown). The dynamic linking implant 100 may be positioned on the polyaxial heads 112 and 113 such that the dynamic linking implant 100 is allowed to float (free to move) to establish a natural height or position of the dynamic linking implant 100. In certain embodiments the natural position of the dynamic linking implant 100 may allow an axis of a pivot point of a first and second link members 125 and 126 and a center axis of one or more attachment brackets 120 and 122 to converge toward a common area “A” located between a disc of the vertebrae to which the pedicle screws are attached. A height adjustment mechanism 130 and 132 may then be used to lock or secure the height or position of the dynamic linking implant 100 while still allowing the dynamic linking implant to rotate or move about the common area “A”.

Dynamic linking implants 1 or 100 may incorporate different biocompatible materials, for example the various components may be manufactured from polymers such as PEEK or UHMWPE. Filled materials may be used such as carbon filled peek. Various metals may also be used such as stainless steel, nitinol or titanium. Bearing or moving surfaces, for example the first ends 20 and 36 may be manufactured from cobalt chrome or may be chrome plated. Surfaces that bear on one another may be manufactured from different materials to reduce wear and friction, for example, a carbon filled PEEK surface of one component may act as a bearing against a cobalt chrome surface of another component. Dynamic linking implant 1 or 100 may also have an elastomeric or fabric covering (not shown).

What is claimed is:

1. A dynamic stabilization spinal implant comprising:
a first link member including:
a first end portion;
a second end portion including a first stop and a second stop;
a first height adjustment mechanism coupling the first end portion to the second end portion, wherein the first height adjustment mechanism includes a first body member having first passage and a second passage generally transverse to the first passage, a first wedge member positioned within the first passage and a second wedge member secured between the first wedge member and the first end portion;
a second link member coupled to the first link member, wherein the second link member includes:
a third end portion
a fourth end portion positioned within and pivotably coupled to the second end portion;
a second height adjustment mechanism including a second body member having third passage and a fourth passage generally transverse to the third passage, a third wedge member positioned within the third passage and a fourth wedge member secured between the third wedge member fastener and the third end portion; and
a force control mechanism having a main body coupled to the second link member, an extension control member and a flexion control member each including a plurality of successive wave elements in which the wave elements include alternating curved crest and curved trough portions wherein the extension control member extends from the main body towards the first stop and the flexion control member extends from the main body towards the second stop.

2. The dynamic stabilization spinal implant of claim 1 wherein the first body member is positioned at least partially within the first end portion.

3. The dynamic stabilization spinal implant of claim 1 wherein the second body member is positioned at least partially within the third end portion.

4. The dynamic stabilization spinal implant of claim 1 wherein the second arm further comprises a recess and the main body of the force control mechanism further comprises a slot and an adjustment member positioned within the slot of the main body and the recess of the first arm.

5. The dynamic stabilization spinal implant of claim 4 wherein the main body has a first position wherein the adjustment member is bias towards a first end of the slot and a second position wherein the adjustment member is bias towards a second end of the slot.

6. The dynamic stabilization spinal implant of claim 5 wherein the flexion control member is compressed in the first position.

7. The dynamic stabilization spinal implant of claim 5 wherein the extension control member is compressed in the second position.

8. The dynamic stabilization spinal implant of claim 1 further comprising a pin coupling the fourth end portion the second end portion.

9. The dynamic stabilization spinal implant of claim 8 wherein the first and second link members pivot about the pin.

10. A dynamic stabilization spinal implant comprising:
a first link member having a first end portion and a second end portion including a first stop and a second stop circumferentially spaced apart from the first stop;
a second link member having a third end portion and a fourth end portion positioned within and pivotably coupled to the second end portion; and
a force control mechanism coupled to the first and second link members and including:
a main body coupled to the second link member,
an extension control member extending from the main body towards the first stop and having a first plurality of successive wave elements in which include one or more alternating curved crest and curved trough portions
a flexion control member extending from the main body towards the second stop and having a second plurality of successive wave elements which the include one or more alternating curved crest and curved trough portions.

11. The dynamic stabilization spinal implant of claim 10 wherein the flexion control member is compressed when the first link member pivots relative to the second link member.

12. The dynamic stabilization spinal implant of claim 10 wherein the extension control member is compressed when the first link member pivots relative to the second link member.

13. The dynamic stabilization spinal implant of claim 10 wherein the extension control member is in a compressed position when the flexion control member is in a neutral position.

14. A method of stabilizing a pair of adjacent boney structures with a dynamic linkage comprising the steps of:
rotating a first link member and second link member relative to each other and about a common pivot point,
compressing a first plurality of successive wave elements in which the wave elements include one or more alternating curved crest and curved trough portions to apply a first force between the first and second link members as the first and second arms rotate in a clockwise direction; and
compressing a second plurality of successive wave elements which include one or more alternating curved crest and curved trough portions to apply a second force between the first and second arms as the first and second arms rotate in a counterclockwise direction.

15. The method of claim 14 further comprising the step of decreasing the first force as the second force is applied.

16. The method of claim 14 wherein the first and second forces are unequal.
